(19) United States
(12) Patent Application Publication
(10) Pub. No.: US 2008/0160088 A1
(43) Pub. Date: Jul. 3, 2008

(54) COMPOSITION CONTAINING HYDROXYAPATITE AND A CALCIUM SALT

(75) Inventor: Vanessa MACKOWIAK, Antony (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/684,149

(22) Filed: Mar. 9, 2007

Related U.S. Application Data
(60) Provisional application No. 60/787,167, filed on Mar. 30, 2006.

(30) Foreign Application Priority Data
Mar. 23, 2006 (FR) 06 51008

(51) Int. Cl. A61K 9/14; A61K 33/42; A61P 17/18

(52) U.S. Cl. 424/489; 424/602

(57) ABSTRACT

Composition containing, preferably in a physiologically acceptable medium, at least hydroxyapatite that is at least partially in free form and at least one calcium salt. Method of use. The invention method and composition is preferably used for preventing and/or reducing the embrittlement of the skin and/or semi-mucous membranes and/or for protecting the skin and/or semi-mucous membranes against water loss and/or external attack

### REFERENCE TO PRIOR APPLICATIONS

This application claims priority to U.S. provisional application 60/787,167 filed Mar. 30, 2006, and to French patent application 0651008 filed Mar. 23, 2006, both incorporated herein by reference.

### FIELD OF THE INVENTION

The invention relates to a composition comprising at least hydroxyapatite and at least one calcium salt. The hydroxyapatite is preferably at least partially in free form. The calcium salt is preferably calcium pantetheine sulfonate.

The invention also relates to a cosmetic care process, especially for mature or even very mature skin, using this composition and directed especially towards improving the barrier function of the skin and/or semi-mucous membranes and thus towards promoting their protection against water loss and external attack.

The invention also relates to a care composition, in particular for mature skin, or even very mature skin, which is intended to improve and/or reinforce the barrier function of the skin and/or semi-mucous membranes.

The invention especially relates to a composition comprising, in a physiologically acceptable medium, at least hydroxyapatite and at least one calcium salt.

The invention also relates to a cosmetic care process, especially for mature or even very mature skin, using this combination and directed especially towards improving the barrier function of the skin and/or semi-mucous membranes and thus towards promoting their protection against water loss and external attack.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Human skin consists of two compartments, namely a deep compartment, the dermis, and a superficial compartment, the epidermis.

The epidermis is in contact with the external environment. One of its roles consists in protecting the body against dehydration and external attack, especially associated with environmental factors such as irritants or pollutants (detergents, pollution, cigarette smoke, etc.), mechanical stresses (rubbing, abrasion, shaving, frequent washing, etc.), thermal or climatic imbalances (cold, wind, dryness, UV radiation, etc.), xenobiotics (microorganisms, allergens, etc.), cosmetic or dermatological chemical treatments (scrubbing, antiacne treatment, etc.) or physiological factors (age, stress, etc.).

It is mainly composed of three types of cell: keratinocytes, which are in the vast majority, melanocytes and Langerhans cells, which are delimited by an intercellular lipid structure. This lipid structure and the cohesion of the epidermal cells participate in the water exchanges of the skin and in the skin's protective function or “barrier function”, on the one hand with regard to water loss (transepidermal water loss) and on the other hand with regard to external attack.

The barrier function of the skin and/or semi-mucous membranes may be impaired especially by internal physiological factors such as age, stress or hormonal changes and/or by environmental factors such as thermal or climatic imbalances, xenobiotics, irritants or mechanical stresses such as rubbing, washing, shaving, etc.

The skin and/or semi-mucous membranes become embrittled, become drier, more subject to cracking and chapping, less resistant to mechanical friction and more permeable to the external environment and to bacteria.

On the skin and/or the lips, the appearance of dryness and/or microchaps may have the consequence of reducing the homogeneity of makeup applied thereto (e.g.: foundation, lipstick), which may not be aesthetic.

Impairment of the barrier function of the skin and/or semi-mucous membranes increases with age, especially after the age of 40 and in particular after the age of 50, and this is all the more true after the age of 60-65, both for women and men, and in particular women.

It is known, for example, that a hormonal decrease during the menopause results in a reduction in the level of cutaneous lipids. The skin then becomes drier, more fragile and more subject to cracking and chapping.

General disorganization of the cutaneous structure takes place, associated with a reduction in the cohesion between the cells, at both the epidermal and dermal levels.

Besides aged skin and in particular mature or even very mature skin, other types of skin show a reduced cutaneous barrier function. They are especially:
sensitive skin,
dry skin whose protective hydrolipid film is modified or impaired.

To improve and/or reinforce the barrier function of the skin and/or semi-mucous membranes, one conventional solution consists in permanently supplying external lipids (e.g.: fatty substances, ceramides) and/or agents that promote the synthesis of epidermal lipids (e.g.: vitamin C and derivatives thereof) and/or emollients or moisturizers.

However, there is still a need to find novel compositions for improving the cellular cohesion and/or for reinforcing the barrier function of the skin and/or semi-mucous membranes, in particular compositions that are suited to caring for mature skin or even very mature skin.

### SUMMARY OF THE INVENTION

The inventor has shown that hydroxyapatite combined with at least one calcium salt satisfies this need. Preferably, the hydroxyapatite is in at least partially in free form and/or in the form of particles of 1 to 10 μm. The inventor has shown that calcium salts can potentiate and/or increase the effect of hydroxyapatite on improving the barrier function of the skin. Preferably, the at least one calcium salt comprises calcium pantetheine sulfonate.

The combination of hydroxyapatite with at least one calcium salt also makes it possible to reduce the amount of hydroxyapatite needed to obtain the desired effect on the skin and/or semi-mucous membranes, namely an effect on improving the barrier function.

### DETAILED DESCRIPTION OF THE PREFERRED EMBODIMENTS

According to the invention, the term “semi-mucous membranes” includes the lips, the term “mature skin” means the skin of individuals at least 40 years old, the term “very mature skin” means the skin of individuals at least 50 years old, including in particular at least 60 years old and even 65 years old.

The use of hydroxyapatite in the form of porous particles of 1 to 10 μm in moisturizing or cleansing cosmetic compositions is known from patent application WO 96/41611: the hydroxyapatite in the form of porous particles combined with moisturizing and antimicrobial agents, serves as a vector and makes it possible especially to promote the absorption, transport and release of these active agents onto the skin.

However, to the inventor's knowledge, it has never been suggested hitherto to combine hydroxyapatite, especially at least partially in free form, with a calcium salt to potentiate and/or increase their effect, in particular to improve the cohesion of cells and/or to reinforce the barrier function of the skin and/or semi-mucous membranes.

The expression “hydroxyapatite at least partially in free form” used according to the invention means hydroxyapatite that is at least partially not combined and/or not complexed with another powder of organic, mineral or metallic type, as opposed to the form of hydroxyapatite that constitutes a composite powder within the meaning of patent application FR 2 594 130.

FR 2 594 130 describes a composite powder in which an organic or mineral powder forming the core is substantially completely covered with another type of powder, such as a hydroxyapatite powder. This composite form is intended to improve the surface characteristics of the powder, in particular its capacity to absorb sebum waste and its covering power, compared with hydroxyapatite that is at least partially in free form.

The hydroxyapatite used according to the present invention is preferably hydroxyapatite at least partially in free form, in particular at least 50% in free form, preferably at least 70% and more preferentially at least 90% in free form, and better still 100% in free form.

The invention thus relates especially to a composition comprising, in a physiologically acceptable medium, at least hydroxyapatite at least partially in free form and at least one calcium salt.

### Hydroxyapatite

Hydroxyapatite is a calcium phosphate with a calcium/phosphorus mole ratio ranging from 0.5 to 20, having an apatite structure (Fragrance Journal, pages 144 to 148, January 1999).

It is present in the natural state in the ground substance of bone extracellular matrix and the teeth. It is in crystalline form, i.e. with an ordered sequence of the atoms in space. The hydroxyapatite crystals are visible by electron microscope, between the collagen fibres or within them and have the appearance of elongate needles.

Hydroxyapatite has the empirical formula [Ca₁₀(PO₄)₆(OH)₂] or 3Ca₃(PO₄)₂ Ca(OH)₂.

Hydroxyapatite may be of natural or synthetic origin. It may especially be obtained synthetically by reaction between a calcium hydroxide and a phosphoric acid.

Preferably, the hydroxyapatite used according to the invention is at least partially in free form, in particular at least 50% in free form, preferably at least 70% and more preferentially at least 90% in free form, and better still 100% in free form.

In another preferred embodiment, the hydroxyapatite used in the composition of the invention is present in the form of particles with a numerical mean size of less than or equal to 50 μm, especially ranging from 0.1 μm to 50 μm, preferably less than or equal to 20 μm, especially ranging from 0.1 μm to 20 μm and preferentially less than or equal to 10 μm, especially ranging from 0.1 μm to 10 μm.

Preferably, the hydroxyapatite used according to the invention is in the form of particles with a numerical mean size ranging from 1 to 10 μm, preferably from 2 to 6 μm and even more preferentially with a size of 4 μm.

The term “numerical mean size” means the mean diameter of a population of particles. This mean diameter may be determined by transmission electron microscopy or from measuring the specific surface area via the BET method, or alternatively by means of a laser granulometer.

In a highly preferred embodiment the hydroxyapatite used according to the invention is at least partially in free form and is present in the form of particles.

Preferably, the hydroxyapatite represents at least 5% to 99+% by weight relative to the total weight of the particles, including 10, 20, 30, 40, 50, 60, 70, 80, 90 and 100%, including all values and subranges therebetween. Preferably, the hydroxyapatite according to the invention is present in a sufficient amount in combination with the calcium salt to obtain the desired beneficial effect(s) on the skin and/or semi-mucous membranes, in particular an effect on improving the barrier function. This amount may be evaluated, for example, on a skin model as described in the examples below.

Preferably, the hydroxyapatite represents at least 50% by weight relative to the total weight of the particles, better still at least 70% by weight relative to the total weight of the particles and even more preferentially at least 90% by weight relative to the total weight of the particles.

In particular, hydroxyapatite in the form of substantially pure hydroxyapatite particles will be used, i.e. particles comprising at least 99% by weight of hydroxyapatite relative to the total weight of the particles.

Examples of such hydroxyapatite particles are described in patent application WO 96/41611. They may be obtained by aggregating hydroxyapatite crystals with a mean size ranging from 0.05 to 0.10 μm, forming spherical particles that are then sintered at high temperature to obtain porous spherical particles that are mechanically, physically and chemically stable. The numerical mean diameter of these particles ranges from 1 to 10 μm and preferably from 2 to 6 μm.

While not bound by theory, it is believed that these particles allow the active agents of the composition to be absorbed, transported and subsequently released onto the skin. Useful spherical porous particles are sold, for example, under the name Hydroxysomes™ by the company Laboratory Skin Care (LSC).

The amount of hydroxyapatite in the invention compositons is not limited and may preferably represent from 0.001% to 10% by weight relative to the total weight of the composition, preferably from 0.01% to 5% by weight and even more preferentially from 0.05% to 1% by weight relative to the total weight of the composition, including 0.005, 0.03. 0.06, 0.07, 0.2, 0.4, 0.6, 0.8, 2, 3, 4, 5, 6, 7, 8 and 9% by weight relative to the total weight of the composition, including all values and subranges therebetween, will be used.

### Calcium Salts

As calcium salts that may be used in the compositions of the invention in combination with the hydroxyapatite as described above, organic calcium salts or mineral calcium salts may preferably be used.

Examples of “organic calcium salts” that may be particularly mentioned include calcium acetate, ascorbate, aspartate, benzoate, citrate, gluconate, lactate, pantetheine sulfonate, pantothenate, proprionate or pidolate salts or calcium PCA, and mixtures thereof. A preferred organic salt is calcium pantetheine sulfonate.

Examples of “mineral calcium salts” that may be particularly mentioned include calcium carbonate, chloride, phosphate, silicate or sulfate, and mixtures thereof. A preferred mineral salt is calcium chloride.

An organic calcium salt will be used in particular. More than one calcium salt can be used, and when more than one are present it may include any combination of organic calcium salts and/or mineral calcium salts. That is, both organic calcium salt(s) and mineral calcium salt(s) may be present.

According to one preferred mode of the invention, a calcium pantetheine sulfonate is used, of formula

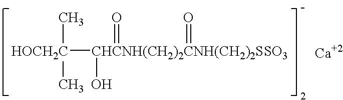

also known as calcium pantetheine sulfonate or calcium bis(N-pantothenyl-β-aminoethyl) thiosulfate.

Calcium D-pantetheine-S-sulfonate 70, also known as calcium bis(N-pantothenyl-β-aminoethyl) thiosulfate, in the form of an aqueous solution containing 70% active material, sold by the company Sogo Pharmaceutical can be used, for example.

The amount of calcium salt that may be used in the composition of the invention in combination with the hydroxyapatite is not limited and depends on the beneficial effect desired on the skin and/or semi-mucous membranes, and in particular depends on the desired effect on improving the barrier function.

By way of example, the amount present in the composition according to the invention will range from 0.001% to 10% by weight relative to the total weight of the composition, preferably from 0.01% to 5% by weight relative to the total weight of the composition and even more preferentially from 0.05% to 1% by weight relative to the total weight of the composition, including 0.005, 0.03. 0.06, 0.07, 0.1, 0.2, 0.4, 0.6, 0.8, 2, 3, 4, 5, 6, 7, 8 and 9% by weight relative to the total weight of the composition, including all values and subranges therebetween.

In one preferred embodiment the hydroxyapatite and the calcium salt are present in the composition in a concentration ratio ranging from 10:1 to 1:10 and preferably from 10:1 to 1:5.

In one preferred embodiment the hydroxyapatite and the calcium salt will be used in a concentration ratio ranging from 7:1 to 3:1 and even more preferentially in a concentration ratio of 5:1.

The composition according to the invention may also contain at least one active agent whose effect is complementary to the combination according to the invention, such as at least one compound chosen from desquamating agents and/or moisturizers; agents for stimulating the synthesis of dermal macromolecules and/or for preventing their degradation; agents for stimulating fibroblast or keratinocyte proliferation and/or keratinocyte differentiation; agents for promoting the synthesis of epidermal lipids; epidermal lipids, in particular ceramides; antioxidants and free-radical scavengers, and mixtures thereof.

The following lists describe preferred materials in preferred amounts. However, the active agents useful herein are not limited thereto.

Desguamating Agents and/or Moisturizers

Examples of desquamating agents and/or moisturizers that may be mentioned include β-hydroxy acids, in particular salicylic acid and derivatives thereof (including 5-n-octanoylsalicyclic acid); α-hydroxy acids, such as glycolic acid, citric acid, lactic acid, tartaric acid, malic acid or mandelic acid, and mixtures thereof; urea; gentisic acid; oligofucoses; cinnamic acid; extracts of Saphora japonica; resveratrol and certain jasmonic acid derivatives.

A preferred desquamating agent is 5-n-octanoylsalicyclic acid.

These desquamating agents and/or moisturizers may represent from 0.01% to 5% and preferably from 0.1% to 1% of the total weight of the composition according to the invention.

Agents for Stimulating the Synthesis of Dermal Macromolecules and/or for Preventing Their Degradation

Among the active agents for stimulating dermal macromolecules or for preventing their degradation, mention may be made of those that act:
either on collagen synthesis, such as extracts of Centella asiatica; asiaticosides and derivatives; ascorbic acid or vitamin C and derivatives thereof; synthetic peptides such as iamin, biopeptide CL or palmitoyloligopeptide sold by the company Sederma; palmitoyl pentapeptide-3 or Matrixyl; peptides extracted from plants, such as the soybean hydrolysate sold by the company Coletica under the trade name Phytokine®; and plant hormones such as auxins and lignans;
or on the inhibition of collagen degradation, in particular agents acting on the inhibition of metalloproteases (MMP), more particularly such as MMP 1, 2, 3 and 9. Mention may be made of: retinoids and derivatives, oligopeptides and lipopeptides, lipoamino acids, the malt extract sold by the company Coletica under the trade name Collalift®; extracts of mulberry or of rosemary; lycopene; isoflavones, derivatives thereof or plant extracts containing them, in particular extracts of soybean (sold, for example, by the company Ichimaru Pharcos under the trade name Flavosterone SB®), of red clover, of flax, of kakkon or of sage; the dipalmitoyl hydroxyproline sold by SEPPIC under the name Sepilift DPHP®; Baccharis genistelloide or Baccharine sold by Silab;
or on the synthesis of molecules belonging to the elastin family (elastin and fibrillin) such as: retinol and derivatives; the extract of Saccharomyces Cerivisiae sold by the company LSN under the trade name Cytovitin®; and the extract of the alga Macrocystis pyrifera sold by the company Secma under the trade name Kelpadelie®; and the N-acylamino amide compounds described in patent application WO 01/94381, such as {2-[acetyl(3-trifluoromethylphenyl)amino]-3-methylbutyrylamino}acetic acid, also known as N-[N-acetyl N′-(3-trifluoromethyl)phenyl valyl]glycine or N-acetyl-N-[3-(trifluoromethyl)phenyl]valyl glycine or acetyl trifluoromethyl phenyl valyl glycine;
or on the inhibition of elastin degradation, such as the peptide extract of Pisum sativum grains sold by the company LSN under the trade name Parelastyl®; heparinoids; and the N-acylamino amide compounds described in patent application WO 01/94381, such as {2-[acetyl(3-trifluoromethylphenyl)amino]-3-methylbutyrylamino}acetic acid, also known as N-[N-acetyl N′-(3-trifluoromethyl)phenyl valyl]glycine or N-acetyl-N-[3-(trifluoromethyl)phenyl]valyl glycine or acetyl trifluoromethyl phenyl valyl glycine.

A preferred agent acting on collagen synthesis is a soybean hydrolysate, such as the product sold by the company Coletica under the trade name Phytokine®.

A preferred agent acting on the synthesis of molecules belonging to the elastin family is an extract of Saccharomyces Cerivisiae, such as the product sold by the company LSN under the trade name Cytovitin®.

These agents that increase the synthesis of dermal macromolecules and/or that prevent their degradation may represent from 0.01% to 5% and preferably from 0.1% to 1% of the total weight of the composition according to the invention.

Agent for Stimulating Fibroblast or Keratinocvte Proliferation and/or Keratinocyte Differentiation

The agents for stimulating fibroblast proliferation that may be used in the composition according to the invention may be chosen, for example, from plant proteins or polypeptides, especially extracted from soybean (for example a soybean extract sold by the company LSN under the name Eleseryl SH-VEG 8® or sold by the company Silab under the trade name Raffermine®); and plant hormones such as gibberellins and cytokinins.

The agents for stimulating keratinocyte proliferation that may be used in the composition according to the invention especially comprise retinoids such as retinol and esters thereof, including retinyl palmitate; adenosine; phloroglucinol; walnut cake extracts sold by the company Gattefosse; and extracts of Solanum tuberosum sold by the company Sederma.

The agent for stimulating keratinocyte differentiation include a peptide extract of lupin such as the product sold by the company Silab under the trade name Structurine®; sodium β-sitosteryl sulfate, such as the product sold by the company Seporga under the trade name Phytocohesine®; and a water-soluble extract of corn, such as the product sold by the company Solabia under the trade name Phytovityl®; a peptide extract of Voandzeia substerranea, such as the product sold by the company Laboratoires Sérobiologiques under the trade name Filladyn LS 9397®; and lignans such as secoisolariciresinol.

### Agents for Promoting the Synthesis of Epidermal Lipids

Examples that may be mentioned include vitamin C and derivatives thereof, in particular the 2-O-α-D-glucopyranosyl derivative of L-ascorbic acid (or ascorbyl glucoside); cinnamic acid and derivatives thereof; auxins.

### Galenics

The composition according to the invention is preferably suitable for topical application to the skin and/or semi-mucous membranes or suitable for oral administration. Preferably, it will be a composition suitable for topical application to the skin and/or the lips.

This composition may be, for example, a care composition or a makeup composition. It will preferably be a care composition.

The composition may be, for example, a cosmetic or dermatological composition. Preferably, it will be a cosmetic composition, in particular a skin and/or lip care composition.

For oral administration, in particular “oral cosmetic” administration, it may especially be in the form of wafer capsules, gel capsules, coated tablets, granules, chewing gum, gels, drinkable syrups, tablets or any other form known to those skilled in the art. In particular, the combination of the active agents according to the invention may be incorporated into any form of food supplement or of enriched food, for example nutritional bars, or compacted or non-compacted powders. The powders may be dilutable with water, in soda, dairy products or soybean derivatives, or may be incorporated into nutritional bars. The active agents may be formulated with the excipients and components that are common for such oral compositions or food supplements, i.e. especially fatty and/or aqueous components, humectants, thickeners, preserving agents, texture, taste and/or coating agents, antioxidants, preserving agents and colorants that are common in the food sector. The formulating agents and excipients for oral compositions, and especially for food supplements, are known in this field and will not be described in detail herein.

The composition according to the invention preferably comprises a physiologically acceptable medium, i.e. a medium that is compatible with the skin and/or its integuments. It is preferably a cosmetically acceptable medium, i.e. a medium of pleasant colour, odour and feel, which does not cause any unacceptable discomfort (stinging, tautness or redness) liable to put the consumer off using this composition.

For topical application to the skin and/or semi-mucous membranes, the composition according to the invention may be in any galenical form, including those conventionally used for topical application and especially in the form of dispersions of the lotion or aqueous gel type, emulsions of liquid or semi-liquid consistency of the milk type, obtained by dispersing a fatty phase in an aqueous phase (O/W) or conversely (W/O), or suspensions or emulsions of soft, semi-solid or solid consistency of the cream, gel, serum, stick or mask type, or alternatively multiple emulsions (W/O/W or O/W/O), aerosols, microemulsions, vesicular dispersions of ionic and/or nonionic type, or wax/aqueous phase dispersions. These compositions are prepared according to the usual methods.

According to one preferred mode, the composition according to the invention will not be a powder. According to another preferred mode, the composition according to the invention will not be an anhydrous composition.

The composition may also be in the form of a mousse or in the form of a spray or an aerosol, then comprising a propellant under pressure. According to one preferred embodiment of the invention, the composition is in the form of an O/W emulsion (e.g.: cream) or W/O emulsion (e.g.: serum) or a gel of aqueous type (e.g.: lotion).

When the composition is an emulsion, the proportion of the fatty phase of the composition under consideration may range, for example, from 5% to 80% by weight and especially from 5% to 50% by weight relative to the total weight of the composition.

The aqueous phase may consist essentially of water or may comprise a mixture of water and of water-miscible organic solvent (miscibility in water of greater than 50% by weight at 25° C.), for instance lower monoalcohols containing from 1 to 5 carbon atoms, such as ethanol, isopropanol, glycols containing from 2 to 8 carbon atoms such as propylene glycol, ethylene glycol, 1,3-butylene glycol or dipropylene glycol, C₃-C₄ ketones and C₂-C₄ aldehydes.

This aqueous phase (water and optionally the water-miscible organic solvent) may be present in the base composition in a content ranging from 1% to 95% by weight, especially ranging from 3% to 80% by weight and in particular ranging from 5% to 60% by weight relative to the total weight of the base composition.

The fatty phase of the composition may contain fatty or oily compounds, and possibly waxes, gums or pasty fatty substances of plant, animal, mineral or synthetic origin, which may or may not be silicone-based.

As oils that may be used in the composition according to the invention, mention may be made of:
hydrocarbon-based oils of animal origin, such as perhydrosqualene;
hydrocarbon-based oils of plant origin, such as liquid triglycerides of fatty acids containing from 4 to 10 carbon atoms or, for example, plant oils such as apricot kernel oil and shea butter oil;
synthetic esters and ethers, especially of fatty acids, for instance the oils of formulae R¹COOR² and R¹OR² in which R¹ represents a fatty acid residue containing from 8 to 29 carbon atoms, and R² represents a branched or unbranched hydrocarbon-based chain containing from 3 to 30 carbon atoms;
linear or branched hydrocarbons, of mineral or synthetic origin, such as volatile or non-volatile liquid paraffins, and derivatives thereof, isohexadecane, isododecane, petroleum jelly, polydecenes, and hydrogenated polyisobutene such as Parléam® oil;
natural or synthetic essential oils;
branched fatty alcohols containing from 8 to 26 carbon atoms, for instance octyidodecanol;
partially hydrocarbon-based and/or silicone-based fluoro oils, for instance those described in document JP-A-2-295 912;
silicone oils, for instance volatile or non-volatile polymethylsiloxanes (PDMS) containing a linear or cyclic silicone chain, which are liquid or pasty at room temperature, especially cyclopolydimethylsiloxanes (cyclomethicones) such as cyclohexasiloxane and cyclopentasiloxane; polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyl-diphenyltrisiloxanes, 2-phenylethyltrimethyl siloxysilicates and polymethylphenylsiloxanes; and
mixtures thereof.

The other fatty substances that may be present in the oily phase are, for example, fatty acids containing from 8 to 30 carbon atoms, for instance stearic acid, lauric acid, palmitic acid and oleic acid; linear fatty alcohols such as cetyl alcohol and/or stearyl alcohol; pasty fatty substances, for instance lanoline; waxes; and gums such as silicone gums (dimethiconol).

These fatty substances may be chosen in a varied manner by a person skilled in the art so as to prepare a composition having the desired properties, for example in terms of consistency or texture.

This composition may also contain various adjuvants commonly used in cosmetics, such as emulsifiers, including stearic acid, fatty acid esters of polyethylene glycol, fatty acid esters of sorbitan, which are optionally polyoxyethylenated, polyoxyethylenated fatty alcohols and fatty acid esters or ethers of sugars such as sucrose or glucose; preserving agents; sequestrants; fragrances; and thickeners and/or gelling agents, in particular polyacrylamides, acrylic homopolymers and copolymers, and acrylamidomethylpropanesulfonic acid homopolymers and copolymers; fillers; and UVA-active and/or UVB-active photoprotective agents, also known as UV-screening agents.

Examples of fillers that may be mentioned include polyamide (Nylon) particles in spherical form or in the form of microfibres; polymethyl methacrylate microspheres; ethylene-acrylate copolymer powders; expanded powders such as hollow microspheres and especially the microspheres formed from a terpolymer of vinylidene chloride, acrylonitrile and methacrylate and sold under the name Expancel; powders of natural organic materials such as starch powders, especially of corn, wheat or rice starch, which may or may not be crosslinked, such as powders of starch crosslinked with octenylsuccinic anhydride; silicone resin microbeads such as those sold under the name Tospearl by the company Toshiba Silicone; silica; metal oxides such as titanium dioxide or zinc oxide; mica; hollow hemispherical silicone particles such as NLK506 sold by the company Takemoto Oil and Fat; and mixtures thereof.

In particular, the composition according to the invention will comprise at least one UV-screening agent.

As UV-screening agents or UVA-active and/or UVB-active photoprotective agents that may be used in the composition of the invention, mention may be made especially of organic or mineral photoprotective agents.

The organic photoprotective agents are especially chosen from anthranilates; cinnamic derivatives; dibenzoylmethane derivatives; salicylic derivatives; camphor derivatives; triazine derivatives such as those described in patent applications US 4 367 390, EP 863 145, EP 517 104, EP 570 838, EP 796 851, EP 775 698, EP 878 469, EP 933 376, EP 507 691, EP 507 692, EP 790 243 and EP 944 624; benzophenone derivatives; β,β-diphenylacrylate derivatives; benzotriazole derivatives; benzalmalonate derivatives; benzimidazole derivatives; imidazolines; bis-benzazolyl derivatives such as those described in patents EP 669 323 and U.S. Pat. No. 2,463,264; p-aminobenzoic acid (PABA) derivatives; methylenebis(hydroxyphenylbenzotriazole) derivatives as described in patent applications U.S. Pat. No. 5,237,071, U.S. Pat. No. 5,166,355, GB 2 303 549, DE 197 26 184 and EP 893 119; and screening polymers and screening silicones such as those described especially in patent application WO 93/04665; α-alkylstyrene-based dimers such as those described in patent application DE 198 55 649.

The mineral photoprotective agents are chosen from pigments or nanopigments (mean size of the primary particles: generally between 5 nm and 100 nm and preferably between 10 nm and 50 nm) of coated or uncoated metal oxides, for instance nanopigments of titanium oxide (amorphous or crystallized in rutile and/or anatase from), iron oxide, zinc oxide, zirconium oxide or cerium oxide, which are all UV-photoprotective agents that are well known per se. Standard coating agents are moreover alumina and/or aluminium stearate. Such coated or uncoated metal oxide nanopigments are described in particular in patent applications EP 518 772 and EP 518 773.

The photoprotective agents are generally present in the composition according to the invention in proportions ranging from 0.1% to 20% by weight relative to the total weight of the composition and preferably ranging from 0.2% to 15% by weight relative to the total weight of the composition.

Needless to say, a person skilled in the art will take care to select this or these optional additional compound(s) and/or the amount thereof such that the properties of the composition according to the invention are not, or are not substantially, adversely affected by the envisaged addition.

In particular, the composition according to the invention is in a form suitable for topical application to the skin and/or semi-mucous membranes, as described previously.

The topical application of the composition according to the invention is performed according to the usual techniques, for example by application of creams, gels, sera, lotions, sticks or masks onto the skin and/or the lips intended to be treated, in particular onto the lips or onto the skin of the body, the face and/or the neck.

According to one alternative, the composition according to the invention may be in a form suitable for oral administration, as described previously.

The invention also relates to a cosmetic process for caring for the skin and/or semi-mucous membranes, comprising the oral administration and/or the application to the skin, via at least one composition, of the combination of at least hydroxyapatite and of at least one calcium salt.

According to a first process of the invention, the two active agents are conditioned in (i.e., present in, added to, etc.) the same composition, in a form suitable for oral administration or in a form suitable for topical application to the skin and/or semi-mucous membranes.

When the two active agents are conditioned in a topical composition, this composition preferably comprises at least hydroxyapatite that is at least partially in free form and at least one calcium salt.

When the two active agents are conditioned in an oral composition, this composition comprises at least hydroxyapatite and at least one calcium salt.

The invention thus relates to a cosmetic process for caring for the skin and/or semi-mucous membranes, comprising the oral administration or the topical application to the skin of a composition as defined previously.

According to another process of the invention, the two active agents are conditioned in two separate compositions, which may be, respectively, in a form suitable for topical application or in a form suitable for oral administration, or alternatively, for one of the compositions, in a form for topical application, and, for the other composition, in a form for oral administration (mixed kit).

The different compositions according to this process comprise, respectively, at least hydroxyapatite and at least one calcium salt.

When the two compositions are in a form suitable for topical application, they may be conditioned either in two separate application devices or in two compartments of the same application device, allowing either a simultaneous distribution of the two compositions at the time of application to the skin, or a distribution and a separate release of the two compositions.

The invention thus also relates to a cosmetic process for caring for the skin and/or semi-mucous membranes, comprising the oral administration and/or the topical application to the skin, via at least two compositions, of the combination of at least hydroxyapatite and of at least one calcium salt.

In particular, the process according to the invention is intended to improve the moisturization and/or comfort of the skin and/or to reinforce the protection of the skin and/or semi-mucous membranes against external attack.

It is also directed towards preventing and/or reducing the formation of cracking or chapping on the skin and/or semi-mucous membranes.

According to one preferred mode, the composition(s) according to the invention is (are) administered and/or applied to individuals with mature or even very mature skin.

In particular, the composition(s) is (are) intended for individuals of at least 50 or even at least 60-65 years old.

The composition(s) according to the invention may also be administered and/or applied advantageously to sensitive skin and/or dry skin.

The composition(s) may be administered and/or applied daily to the skin and/or semi-mucous membranes, morning and/or evening.

The invention also relates to the use of the combination of at least hydroxyapatite and of at least one calcium salt, as a combination for improving and/or reinforcing the cellular cohesion and/or the integrity of the skin and/or semi-mucous membranes and/or for improving and/or reinforcing the barrier function of the skin and/or semi-mucous membranes.

In particular, the combination and/or the composition according to the invention is intended for preventing and/or reducing the embrittlement of the skin and/or semi-mucous membranes and/or for protecting the skin and/or semi-mucous membranes against water loss and/or external attack, in particular it can be applied to skin and/or semi-mucous membranes in need of:
for protecting against water loss and thus preventing, improving and/or overcoming the external signs of dryness of the skin associated with a deficit of the skin barrier function; and/or
for protecting against external attack, especially associated with environmental factors such as irritants or pollutants (detergents, pollution, cigarette smoke, etc.), mechanical stresses (rubbing, abrasion, shaving, frequent washing of the hands), thermal or climatic imbalances (cold, dryness, radiations), xenobiotics (microorganisms, allergens), cosmetic or dermatological chemical treatments (scrubbing, antiacne treatment, etc.) or physiological factors (age, stress, etc.); and/or for making the skin and/or semi-mucous membranes more resistant to external attack.

According to another mode, the combination and/or the composition according to the invention is intended for improving the comfort of the skin, in particular for preventing and/or reducing tautness and stinging.

According to another aspect of the invention, the combination and/or the composition according to the invention is intended for improving the surface aspect of the skin and/or the lips, in particular the roughness and/or irregularities of the relief.

According to another aspect of the invention, the combination and/or the composition according to the invention is intended for limiting the formation of cracking or chapping on the lips, the hands, the face or the body and thus especially for promoting better homogeneity of a makeup composition subsequently applied to the skin and/or the lips.

The hydroxyapatite and the calcium salt may be conditioned in the same composition or in two separate compositions.

These two complementary separate compositions may be present in a kit or in two compartments of the same application device for simultaneous or sequential release of the compositions, the compositions each possibly being in a form suitable for topical application or in a form suitable for oral administration.

Examples of compositions that are suitable, respectively, for topical application or oral administration are described previously.

According to one preferred mode, a hydroxyapatite that is at least partially in free form will be used.

The invention will now be illustrated by means of the non-limiting examples that follow. In these examples, the amounts are indicated as weight percentages.

### EXAMPLES

### Example 1

### Effect of the Combination of Hydroxyapatite and of a Calcium Salt on the Improvement and/or Reinforcing of the Barrier Function

Hydroxyapatite in the form of particles sold under the name Hydroxysomes™ by LSC and their combination with a calcium D-pantetheine sulfonate sold by SOGO Pharmaceutical were tested on an in vitro model to evaluate the overall effect of this combination on the barrier function of the skin.

This test consists in applying the test active agents to Episkin™ reconstructed epidermides differentiated on D13, and then, after incubation for 5 days, in applying radioactively labelled caffeine and monitoring the diffusion of the labelled caffeine through the reconstructed epidermides.

Vitamin C, which is known to improve the epidermal barrier function via stimulation of the synthesis of the epidermal lipids, is used as a reference molecule for this test (positive control). Water is used as a negative control.

Differentiated (D13) Episkin™ epidermides were placed in 12-well plates containing 2 ml of differentiation medium and were precultured at 37° C. and 5% CO₂ for 24 hours. After incubation, the epidermides were treated topically with 100 μl of each concentration of test product or of the reference product (vitamin C).

Control epidermides were treated topically with 100 μl of water.

The epidermides were then incubated at 37° C. and 5% CO₂ for 5 days, with changing of the medium and new application of the active agents after 2 days.

The treatments were performed in quintuplicate (n=5).

Four epidermides were used for the passage of caffeine and one “control” epidermis was kept to perform histology at the end of the experiment.

After treatment with the test active agents and the reference product (vitamin C), the epidermides were washed and the caffeine was applied to the surface of each epidermis at a rate of 100 μl containing 2 μCi/ml (0.04 mM) of radioactive caffeine and 0.35 mM of cold caffeine, which corresponds to about 500 000 cpm total.

The % activity of caffeine after 2 hours of application was measured.

The results obtained are collated in the following table:

| | | | Reduction of the |
|---|---|---|---|
| | | % activity | passage of caffeine |
| | | relative to | after 2 hours of |
| TREATMENT | CONCENTRATION | the control | application (in %) |
| Vitamin C | 200 μg/ml | 59 | −41% |
| Hydroxyapatite* | 50 μg/ml | 64 | −36% |
| Hydroxyapatite | 5 μg/ml | 92 (NS) | NS |
| Hydroxyapatite + Calcium | 5 μg/ml and | 60 | −40% |
| Pantetheine | 0.003% | | |
| Sulfonate | | | |

(NS = not significant)
*= Hydroxysomes ™ sold by LSC

In parallel and under the same test conditions, the effect of calcium D-pantetheine sulfonate alone at 0.03% and 0.003% on the barrier function of the skin was evaluated.

After 2 hours of application, only the higher concentration (0.03%) showed a significant reduction in the diffusion of caffeine (−24% relative to the control). The result was not significant for the 0.003% concentration.

The results obtained show that vitamin C (200 μg/ml), used as reference molecule, significantly slows down the passage of caffeine (−41% relative to the control, p<0.01). These results validated the test.

Hydroxyapatite tested alone at 50 μg/ml slows down the diffusion of caffeine (−36% of passage, p<0.05). At 5 μg/ml, this active agent shows no effect.

The mixture of hydroxyapatite 5 μg/ml and calcium pantetheine sulfonate 0.003% significantly reduces the passage of caffeine (−40%, p<0.05).

These results show that the effect of hydroxyapatite appears to be potentiated by the calcium pantetheine sulfonate, which at 0.003% had no effect alone on the skin barrier function, and that this effect is similar to that observed with vitamin C.

The results obtained for this combination of active agents reflect an improvement and/or reinforcement in the epidermal barrier function.

### Example 2

### Formulation

Oil-in-water emulsion

| | |
|---|---|
| Calcium pantetheine sulfonate* | 0.10% |
| Hydroxyapatite** | 0.50% |
| Oils | 6.00% |
| Stearyl alcohol | 0.50% |
| UV-screening agents | 10.50% |
| Cyclopentasiloxane | 7.50% |
| Triethanolamine | 1.60% |
| Glycerol | 7.00% |
| Emulsifiers | 5.80% |
| Fragrances | 0.30% |
| Preserving agents | 1.20% |
| Water | qs 100% |

*= calcium bis(N-pantothenyl-β-aminoethyl) thiosulfate as an aqueous 70% solution sold by Sogo Pharmaceutical
**= Hydroxysomes ™ sold by LSC

After application to the skin, the skin is visibly more supple and hydrated.

The above written description of the invention provides a manner and process of making and using it such that any person skilled in this art is enabled to make and use the same, this enablement being provided in particular for the subject matter of the appended claims, which make up a part of the original description and including a composition comprising, preferably in a physiologically acceptable medium, at least hydroxyapatite that is at least partially in free form and at least one calcium salt.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The invention method and composition is preferably used by subjects desirous of the benefits noted herein, subjects “in need of” these benefits. Such subjects are typically desirous of preventing and/or reducing the embrittlement of the skin and/or semi-mucous membranes and/or for protecting the skin and/or semi-mucous membranes against water loss and/or external attack. In particular the invention composition can be applied to skin and/or semi-mucous membranes in need of protecting against water loss and thus preventing, improving and/or overcoming the external signs of dryness of the skin associated with a deficit of the skin barrier function; and/or for protecting against external attack, especially associated with environmental factors such as irritants or pollutants (detergents, pollution, cigarette smoke, etc.), mechanical stresses (rubbing, abrasion, shaving, frequent washing of the hands), thermal or climatic imbalances (cold, dryness, radiations), xenobiotics (microorganisms, allergens), cosmetic or dermatological chemical treatments (scrubbing, antiacne treatment, etc.) or physiological factors (age, stress, etc.); and/or for making the skin and/or semi-mucous membranes more resistant to external attack.

Naturally, one using the invention as disclosed will use an amount of the invention composition effective to obtain the desired benefits. Such amount is inclusive of an amount of the compositions described herein at the disclosed concentrations of active ingredients sufficient to cover the area of the skin, etc. being treated in a single application, and of course includes that amount applied upon repeated application, for example on a daily basis over a course of days, weeks, etc. In a preferred embodiment the invention process includes multiple applications of the invention composition to the area(s) of skin, etc. in need of attention.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in a physiologically acceptable medium, hydroxyapatite and at least one calcium salt, wherein said hydroxyapatite is at least partially in free form.

2. The composition according to claim 1, wherein the hydroxyapatite is at least partially present in the form of particles with a numerical mean size ranging from 0.1 to 50 μm.

3. The composition according to claim 1, wherein the hydroxyapatite is at least partially present in the form of particles with a numerical mean size ranging from 1 to 10 μm.

4. The composition according to claim 1, wherein the hydroxyapatite is at least partially in the form of particles comprising at least 99% by weight of hydroxyapatite relative to the total weight of the particles.

5. The composition according to claim 1, wherein the hydroxyapatite is present in the composition in an amount ranging from 0.01% to 5% by weight relative to the total weight of the composition.

6. The composition according to claim 1, wherein the calcium salt is chosen from organic and mineral calcium salts, and mixtures thereof.

7. The composition according to claim 6, wherein the composition comprises at least one organic calcium salt chosen from calcium acetate, ascorbate, aspartate, benzoate, citrate, gluconate, lactate, pantetheine sulfonate, pantothenate or propionate, and mixtures thereof.

8. The composition according to claim 6, wherein the composition comprises at least one mineral calcium salt chosen from calcium carbonate, chloride, phosphate or sulfate, and mixtures thereof.

9. The composition according to claim 1, comprising calcium pantetheine sulfonate.

10. The composition according to claim 1, wherein the calcium salt is present in the composition in an amount ranging from 0.01% to 5% by weight relative to the total weight of the composition.

11. The composition according to claim 1, wherein the hydroxyapatite and the calcium salt are present in the composition in a concentration ratio ranging from 10:1 to 1:5.

12. The composition according to claim 1, further comprising at least one agent chosen from desquamating agents and/or moisturizers; agents for stimulating the synthesis of dermal macromolecules and/or for preventing their degradation; agents for stimulating fibroblast or keratinocyte proliferation and/or keratinocyte differentiation; agents for promoting the synthesis of epidermal lipids; epidermal lipids; antioxidants and free-radical scavengers, and mixtures thereof.

13. The composition according to claim 1, further comprising at least one UV-screening agent.

14. The composition according to claim 1, wherein said composition is in a form suitable for topical application to the skin and/or semi-mucous membranes.

15. The composition according to claim 1, wherein said composition is in a form suitable for oral administration.

16. A process for caring for the skin and/or semi-mucous membranes, comprising oral administration or topical application to the skin and/or the lips via at least two compositions, one comprising hydroxyapatite and one comprising at least one calcium salt.

17. A process for caring for the skin and/or semi-mucous membranes, comprising the oral administration or the topical application to the skin and/or semi-mucous membranes of the composition as defined in claim 1.

18. The process according to claim 17, for improving the moisturization and/or comfort of the skin and/or towards reinforcing the protection of the skin and/or semi-mucous membranes against external attack and comprising the oral administration or the topical application to skin and/or semi-mucous membranes in need thereof.

19. The process according to claim 17 preventing and/or reducing the formation of cracking or chapping on the skin and/or semi-mucous membranes and comprising the oral administration or the topical application to skin and/or semi-mucous membranes in need thereof.

20. The process according to claim 16, wherein the composition is administered and/or applied to individuals with mature skin.

21. The process according to claim 17, for improving and/or reinforcing the cellular cohesion and/or the integrity of the skin and/or semi-mucous membranes and/or for improving and/or reinforcing the barrier function and comprising the oral administration or the topical application to skin and/or semi-mucous membranes in need thereof.

22. The process according to claim 17, for preventing and/or reducing the embrittlement of the skin and/or semi-mucous membranes and/or for protecting the skin and/or semi-mucous membranes against water loss and/or external attack and comprising the oral administration or the topical application to skin and/or semi-mucous membranes in need thereof.

23. The process according to claims 17, wherein the hydroxyapatite and the calcium salt are present in a single composition that is administered or applied.

24. The process according to claims 17, wherein the hydroxyapatite and the calcium salt are present in separate compositions that are administered or applied.
